(19) United States
(12) Patent Application Publication
(10) Pub. No.: US 2012/0078290 A1
(43) Pub. Date: Mar. 29, 2012

(54) LAPAROSCOPIC INSTRUMENT WITH ATTACHABLE END EFFECTOR

(76) Inventors: Rudolph H. NOBIS, Mason, OH (US); James T. Spivey, Cincinnati, OH (US); Christopher J. Hess, Cincinnati, OH (US); Kevin M. Huey, Cincinnati, OH (US)

(21) Appl. No.: 12/889,454

(22) Filed: Sep. 24, 2010

(51) Int. Cl. A61B 17/28

(52) U.S. Cl. 606/206

(57) ABSTRACT

A laparoscopic surgical device comprises an elongate shaft defining a longitudinal axis, the shaft comprising a distal end and a proximal end. A plurality of arms project distally from the distal end of the elongate shaft, the arms each comprising a lateral notch. The arms are axially slideable relative the elongate shaft and are medially deflectable. An elongate pin is positioned medially relative the arms. The elongate pin is axially slideable relative the arms between a locked position preventing medial deflection of the arms and an unlocked position allowing medial deflection of the arms. A surgical end effector is selectively attachable in vivo and detachable in vivo to the mating feature of the arms, the surgical end effector comprising a torque transfer means and tissue contact apparatus that open and close in response to the axial movement of the two arms when attached to the surgical end effector.

### BACKGROUND

The present invention relates in general to surgical devices and procedures, and more particularly to minimally invasive surgery.

Surgical procedures are often used to treat and cure a wide range of diseases, conditions, and injuries. Surgery often requires access to internal tissue through open surgical procedures or endoscopic surgical procedures. The term “endoscopic” refers to all types of minimally invasive surgical procedures including laparoscopic, arthroscopic, natural orifice intraluminal, and natural orifice transluminal procedures. Endoscopic surgery has numerous advantages compared to traditional open surgical procedures, including reduced trauma, faster recovery, reduced risk of infection, and reduced scarring. Endoscopic surgery is often performed with an insufflatory fluid present within the body cavity, such as carbon dioxide or saline, to provide adequate space to perform the intended surgical procedures. The insufflated cavity is generally under pressure and is sometimes referred to as being in a state of pneumoperitoneum. Surgical access devices are often used to facilitate surgical manipulation of internal tissue while maintaining pneumoperitoneum. For example, trocars are often used to provide a port through which endoscopic surgical instruments are passed. Trocars generally have an instrument seal, which prevents the insufflatory fluid from escaping while an instrument is positioned in the trocar.

While surgical access devices are known, no one has previously made or used the surgical devices and methods in accordance with the present invention.

### SUMMARY

A surgical device is provided that comprises an elongate shaft defining a longitudinal axis, the shaft comprising a distal end and a proximal end. There is a first arm on the elongate shaft comprising a mating feature, the first arm being medially deflectable. A second arm is longitudinally spaced from the first arm comprising a mating feature, the arm being axially slideable relative the elongate shaft and being medially deflectable. An elongate pin is positioned medially relative the second arm, the elongate pin being axially slideable relative the first arm and second arm between a locked position preventing medial deflection of the first arm and second arm and an unlocked position allowing medial deflection of the first arm and second arm. The surgical device further comprises a surgical end effector selectively attachable and detachable to the mating feature of the second arm.

The surgical device may further comprise a lateral notch on the distal end of the first arm and a mating feature on the surgical end effector. The surgical device may have a mating feature comprised of a ring dimensioned to mate with the first arm lateral notch and may further have two or more second arms circumscribing the elongate pin. The surgical device may have two or more first arms and an opening defined by the two or more first arms and an elongate pin having an obturator tip. The surgical device may further comprise a handle operatively connected to the proximal end of the elongate shaft, the handle comprising a trigger controlling the axial movement of the second arm and an actuator controlling the axial movement of the elongate pin, wherein the actuator may be lockable. The end effector may have opposable members that move between open and closed positions in response to axial motion of the second arm.

A laparoscopic surgical device is provided. The device comprises an elongate shaft defining a longitudinal axis, the shaft comprising a distal end and a proximal end and a plurality of first arms on the distal end of the shaft, the first arms each comprising a lateral notch, the arms being medially deflectable. The device further comprises a plurality of second arms positioned medial to the first arms, the second arms projecting distally from the distal end of the elongate shaft and being longitudinally spaced from the first arms, the second arms each comprising a lateral notch, the second arms being axially slideable relative the elongate shaft and being medially deflectable. The device may contain an elongate pin positioned medially relative the second arms, the elongate pin being axially slideable relative the arms between a locked position preventing medial deflection of the arms and an unlocked position allowing medial deflection of the arms. The laparoscopic device further comprises a surgical end effector selectively attachable in vivo and detachable in vivo to the mating features of the first arms and the second arms.

The laparoscopic surgical device of claim may further comprise an obtruator tip on the distal end of the elongate pin and a plurality of first arms defining an opening in the elongate shaft and the opening may extend from the shaft lateral surface through the shaft medial surface. The laparoscopic surgical device further comprises a handle operatively connected to the proximal end of the elongate shaft, the handle comprising a trigger controlling the axial movement of the second arm and an actuator controlling the axial movement of the elongate pin.

Another surgical device is provided where the device comprises an elongate shaft defining a longitudinal axis, the shaft comprising a distal end and a proximal end. The device has a plurality of first arms on the distal end of the shaft, the first arms each comprising a lateral notch, the arms being medially deflectable, the arms defining an opening in the shaft. The surgical device further comprises a plurality of second arms positioned medial to the first arms, the second arms projecting distally from the distal end of the elongate shaft and being longitudinally spaced from the first arms, the second arms each comprising a lateral notch, the second arms being axially slideable relative the elongate shaft and being medially deflectable. The surgical device also comprises an elongate pin positioned medially relative the second arms, the elongate pin being axially slideable relative the arms between a locked position preventing medial deflection of the arms and an unlocked position allowing medial deflection of the arms, the pin having an obturator tip. A surgical end effector is provided for the surgical device, where the end effector is selectively attachable in vivo and detachable in vivo to the mating features of the first arms and the second arms, the surgical end effector comprising a means to manipulate tissue, the tissue manipulation means actuated in response to the axial movement of the second arms when attached to the surgical end effector. The surgical device further comprises a handle operatively connected to the proximal end of the elongate shaft, the handle comprising a trigger controlling the axial movement of the second arm and an actuator controlling the axial movement of the elongate pin.

### BRIEF DESCRIPTION OF DRAWINGS

While the specification concludes with claims which particularly point out and distinctly claim the invention, it is believed the invention will be better understood from the following description taken in conjunction with the accompanying drawings illustrating some non-limiting examples of the invention. Unless otherwise indicated, the figures are not necessarily drawn to scale, but rather to illustrate the principles of the invention.

FIG. 1 depicts surgical procedure with an instrument and loader holding an end effector;

FIG. 2 depicts a close-up view of the distal ends of the instrument and loader in FIG. 1;

FIG. 3 depicts an instrument being inserted into an end effector;

FIG. 3A depicts an isometric cross-sectional view of an end effector;

FIG. 3B depicts an isometric cross-sectional view of an instrument partially inserted into an end effector;

FIG. 3C depicts an end effector with torque arms provided in the lateral surface of the end effector;

FIG. 3D depicts a close up of the end effector of FIG. 3C;

FIG. 3E depicts a cross section of the FIG. 3D end effector with an instrument inserted in the end effector;

FIG. 4 depicts an instrument attached to an end effector being withdrawn from a loader;

FIG. 4A depicts a loader with removable distal end;

FIG. 5 depicts an isometric close-up view of the distal end of an instrument in a locked position;

FIG. 6 depicts an isometric close-up view of the distal end of an instrument in an unlocked position;

FIG. 7 depicts an isometric cross-sectional view of the distal end of an instrument attached to an end effector;

FIG. 7A depicts an isometric cross-sectional view of the distal end of an instrument attached to an end effector with the pin advanced distally;

FIG. 8 depicts an isometric cross-sectional view of the distal end of an instrument attached to an end effector in a pushed-off configuration;

FIG. 9 depicts an instrument handle;

FIG. 10 depicts a bi-polar jawed end effector;

FIG. 11 depicts a cutting shears end effector;

FIG. 12 depicts a Maryland dissector end effector; and

FIG. 13 depicts an ultrasonic shears end effector;

### DETAILED DESCRIPTION

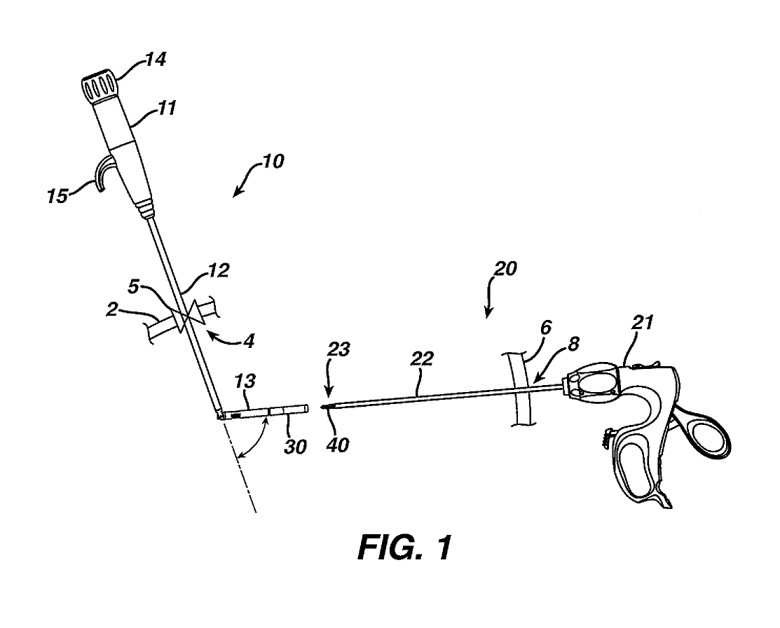

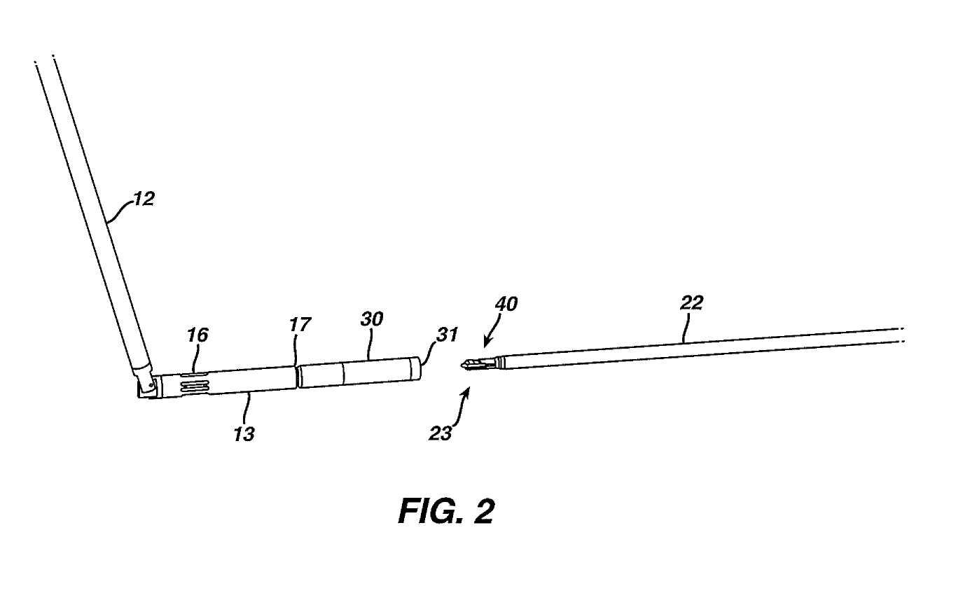

As shown in FIG. 1, instrument (20) comprises an elongate shaft (22) passing through an incision (8) of a tissue wall (6). A loader (10) comprises an elongate shaft (12) passing through an incision (4) of a tissue wall (2). The surgical end effector (30) is selectively attachable in vivo and detachable in vivo to the attachment mechanism (40) located at the distal end (23) of the instrument (20). In this example, the end effector is a jawed tissue grasper, but a variety of other end effectors could be also be used. The end effector (30) may be loaded ex vivo into the distal end (13) of the shaft (12), and then introduced into the surgical field through the incision (4). The loader (10) holds the end effector (30) during the in vivo attachment to and in vivo detachment from the instrument (20). The loader (10) and instrument (20) each includes ex vivo handles (11, 21) attached to the proximal ends of the shafts (12, 22) that enable surgeons to use the devices.

The tissue wall (2, 6) anatomies will vary based on the surgical procedure, but some non-limiting examples include percutaneous incisions into the abdomen, thorax, or pelvis. The incisions (4, 8) may be created with a cutting or puncturing instrument, and will typically be spaced from one another. The tissue walls (2, 6) may be the same or different anatomies. For instance, tissue walls (2, 6) may both be the abdominal wall. In another example, tissue wall (2) could be an organ (e.g., stomach, colon, esophagus, etc.) accessed through a natural orifice, while the incision (8) in tissue wall (6) could be percutaneous. In yet another example, incision (4) may provide access to the abdomen, while the incision (8) may provide access to the pelvis. If pneumoperitoneum is desired, the incisions may include instrument seals, such as those commonly found in trocars. In this example, the instrument seal (5) is schematically shown in incision (4) with the loader (10) passing through the seal (5), while the shaft (22) seals directly with the tissue wall (6) by virtue of the resilience of the tissue without the aid of a sealing device.

The loader shaft (12) in this embodiment is rigid and straight, but the shaft (12) could be curved or flexible, which would be beneficial for natural orifice transluminal introduction of the distal end (13) to the surgical field. The loader (10) may include an articulating distal end (13) controlled by the knob (14). The distal end (13) will typically be introduced and removed through the incision (4) in-line with the shaft (12), and then articulated in vivo to facilitate alignment between the end effector (30) and the shaft (22). The arm (15) is rigidly connected the handle (11) to facilitate grasping of the handle and rotational orientation of the articulated distal end (13) about the shaft (12) axis. In this embodiment, the distal end (13) of the loader (10) comprises a tube opening at the distal tip (17). The tube is dimensioned to receive the end effector (32). The tube (30) includes an engagement feature (16) for holding the end effector (32). While the engagement feature (16) may vary, in this embodiment a plurality of leaf springs provide an interference fit with the end effector (30) to frictionally hold the end effector in the tube. In this embodiment, when the end effector (30) is loaded in the tube, the distal end (32) is positioned in the tube and the proximal end (31) extends from the tube opening (17). This arrangement prevents the jaws of the end effector from opening. After the distal end (23) of the instrument (20) is attached to the proximal end (31) of the end effector (30), the end effector (3) can be pulled from the distal end (13) of the loader (10).

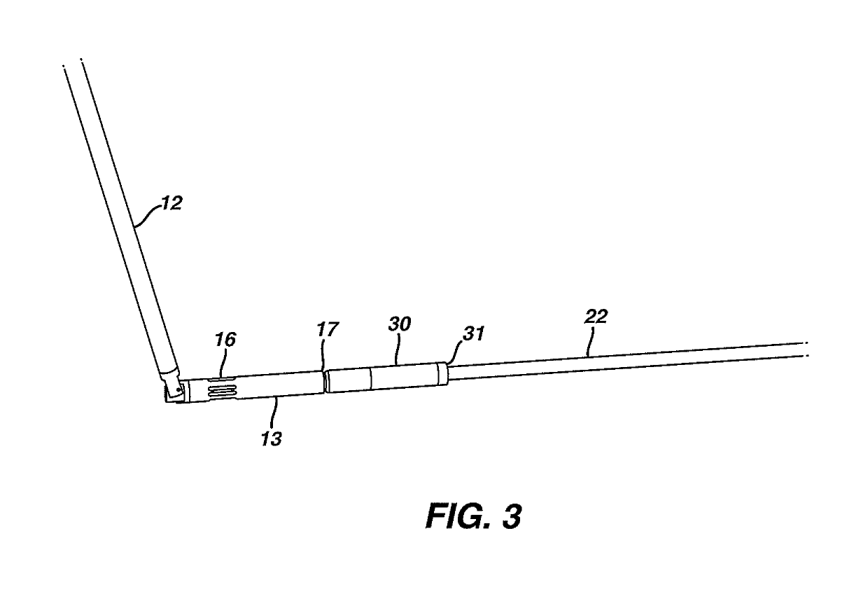

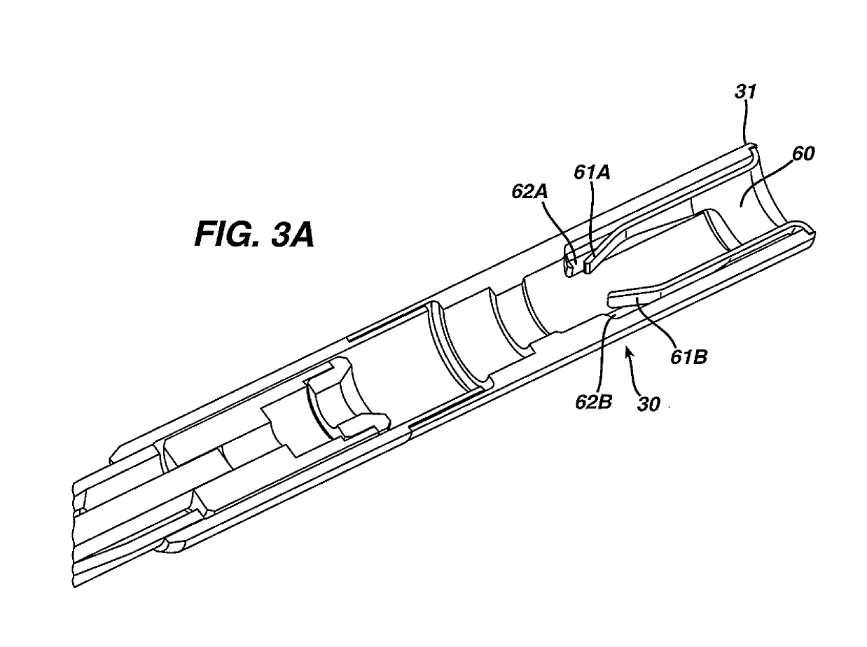

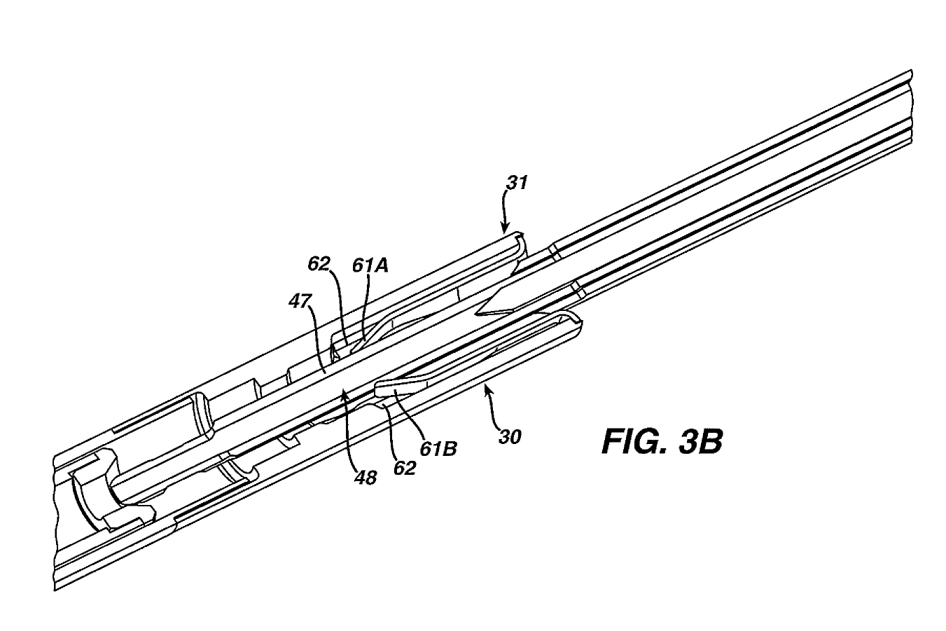

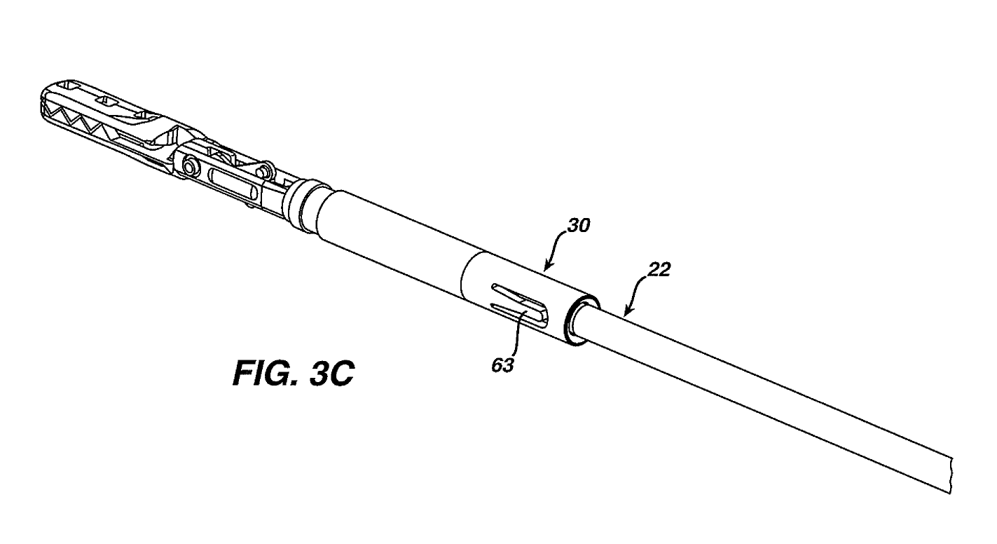

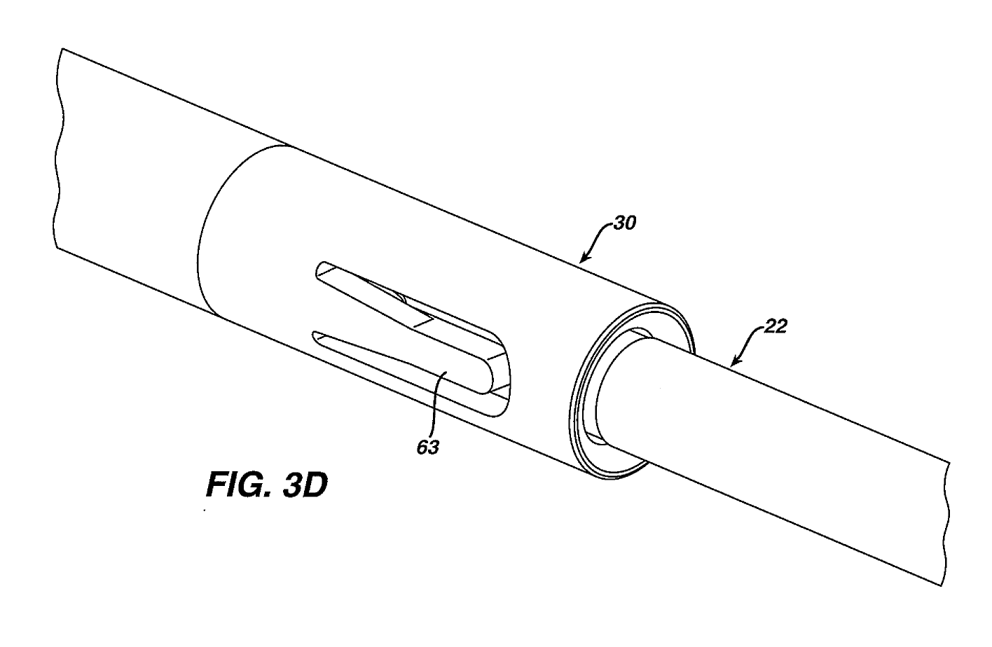

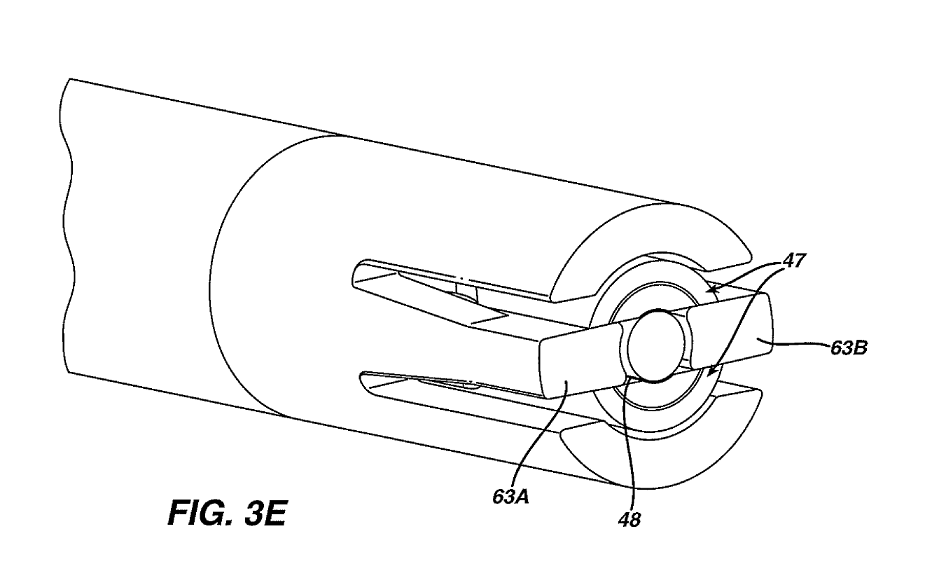

FIG. 3A depicts an example of an end effector provided with a torque key (60). The torque key, in one expression, is fixedly attached to proximal end (31) of end effector (30). Torque key (60) is provided with torque arms (61A, 61B). Torque arms (61) may be provided with a medial angular bend. End effector (30) may also be provided with torque arm recesses (62A, 62B) that permit the torque arms (61) to laterally deflect creating a variable inner diameter of end effector (30). FIG. 3B depicts the instrument shaft (22) partially inserted into end effector (30). In this depiction, torque arms (61) are aligned with flat surfaces on the shaft arms (47) and protrude medially into an opening (48) between shaft arms (47). When shaft (22) is inserted into end effector (30) and the torque arms (61) are not aligned with opening (48), they will remain deflected medially in recess (62) until the shaft (22) is rotated to align torque arms (62) with opening (48). When aligned with the opening (48), torque arms (61) permit transfer of rotational force from the shaft to the end effector.

FIGS. 3C and 3D depict another expression of the end effector (30). The proximal end of the end effector (30) is provided with flexible torque arm (63) formed from the lateral surface of end effector (30). When shaft (22) is inserted into end effector (30), torque arm (63) may deflect laterally where the opening (48) is not aligned with torque arm (63). To facilitate engagement with shaft (22) torque arm (63) may be provided with a chamfered surface. Upon rotation of the shaft (22), the torque arm will align with opening (48). When aligned with the opening (48), torque arm (63) permits transfer of rotational force from the shaft (22) to the end effector (30).

FIG. 3E depicts a cross sectional view of a shaft (22) inserted into end effector (30). In this expression, end effector (30) is provided with two torque arms (63A, 63B). Torque arms (63) are aligned to opening (48) defined by shaft arms (47) creating an interference fit.

In another expression of the surgical instrument, the torque arms (63) may be provided with recessed inner portions that mate with projections on the lateral surface of the shaft (not shown). The shaft projections may be flexible to facilitate entry of the shaft into the end effector. In yet another expression, the end effector may be provided with recesses (not shown) located on the medial surface of the end effector that mate with the projections on the lateral surface of the shaft.

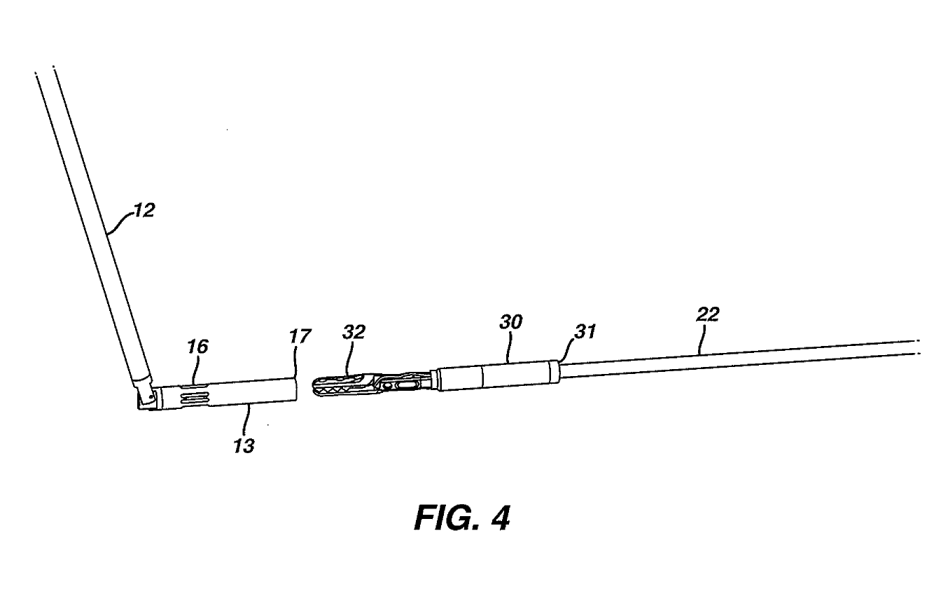

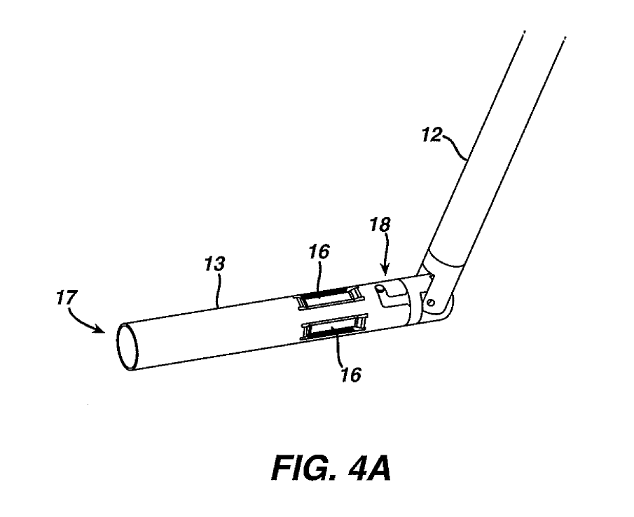

FIG. 4 depicts an instrument (20) attached to an end effector (32) being withdrawn from a loader (13). FIG. 4A depicts an alternative embodiment of a loader (10) where the distal end (13) is selectively attachable and detachable to the shaft (12). As shown in this example, this feature is enabled with a bayonet connection (18), but other connections are also contemplated including snap connections, threaded connections, and the like. One advantage of this alternative embodiment is that different distal end (13) configurations may be used to hold end effectors that may not be accommodated by a single sized tube.

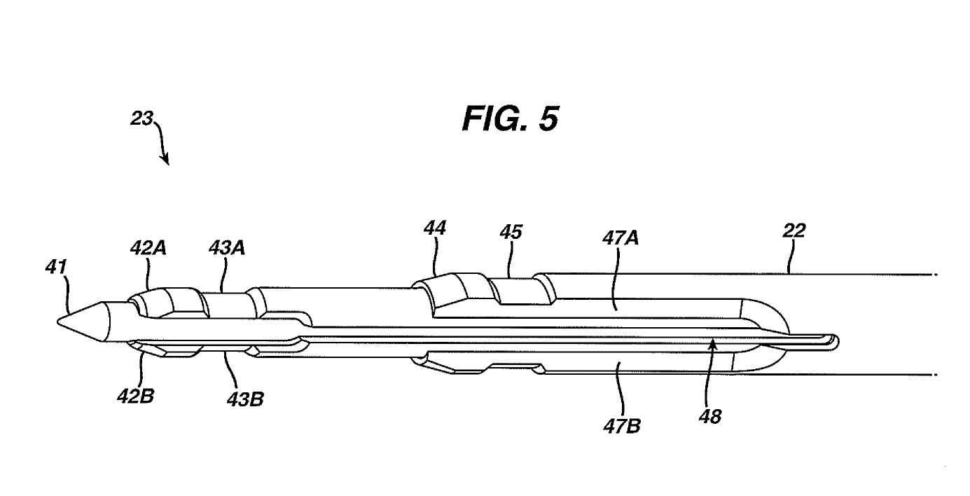

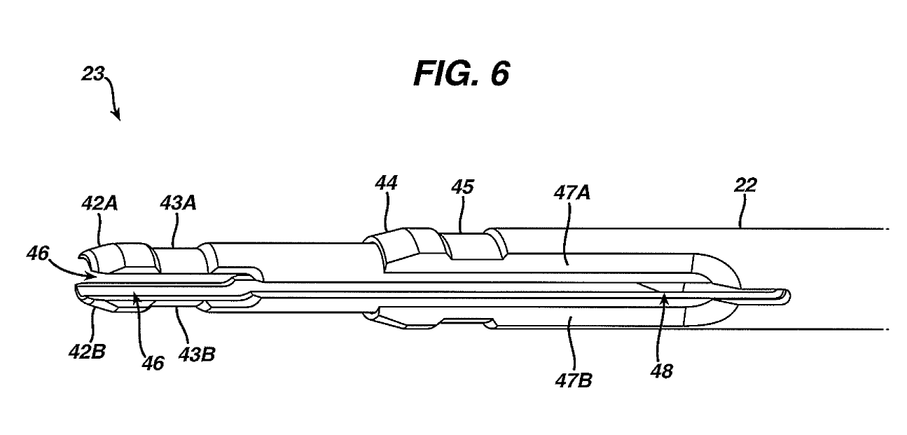

FIGS. 5 and 6 depict a detailed view of one embodiment of an attachment mechanism (40) located at the distal end (23) of the shaft (22). The attachment mechanism (40) comprises a mating feature on the shaft (22), which in this embodiment is a circumferential groove (45) positioned on the lateral surface of shaft arms (47A, 47B). Shaft arms (47A, 47B) may be resiliently flexible into opening (48). The attachment mechanism (40) also comprises second arms (42A, 42B) projecting distally from the distal end (44) of the shaft (22). The second arms may be axially slideable relative the shaft (22) and are resiliently deflectable medially into the gap (46). The second arms each comprise a mating feature, which in this embodiment comprises a stepped lateral notch (43A, 43B). An elongate pin (41) is positioned medially relative the second arms (42) and shaft arms (47) and is axially slideable relative the second arms (42) and shaft arms (47) between a locked position preventing medial deflection of the arms (42 and 47) (an example of which is shown in FIG. 5) and an unlocked position allowing medial deflection of the arms (an example of which is shown in FIG. 6). The pin (41) and second arms (42) may each slide independently relative the shaft (22) and shaft arms (47). FIG. 6 shows the pin (41) fully retracted inside shaft (22) allowing medial deflection of shaft arms (47).

As shown in the embodiment of FIG. 5, the elongate pin (41) may include a pointed obtruator tip. In this configuration the distal end (23) may be used to puncture through the tissue wall (6). The distal ends of the second arms (42) and distal end (44) of the shaft arms (47A, 47B) include tapered surfaces to facilitate passing through the incision (8).

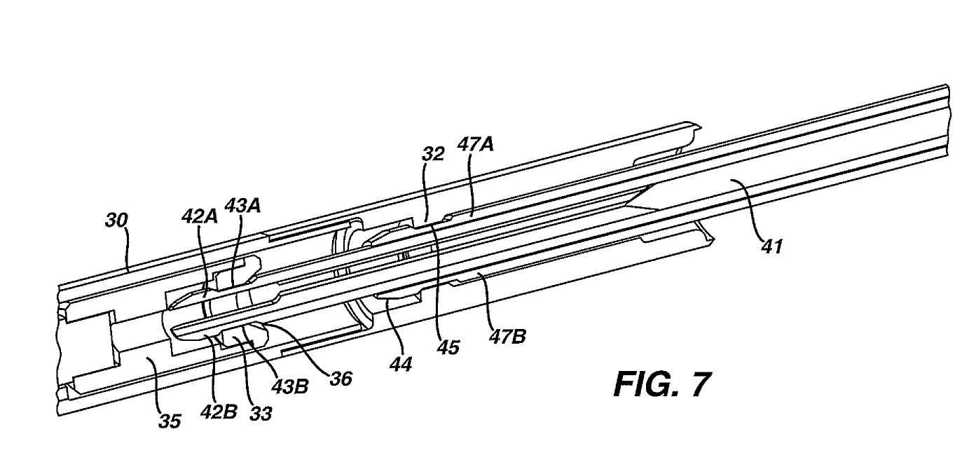

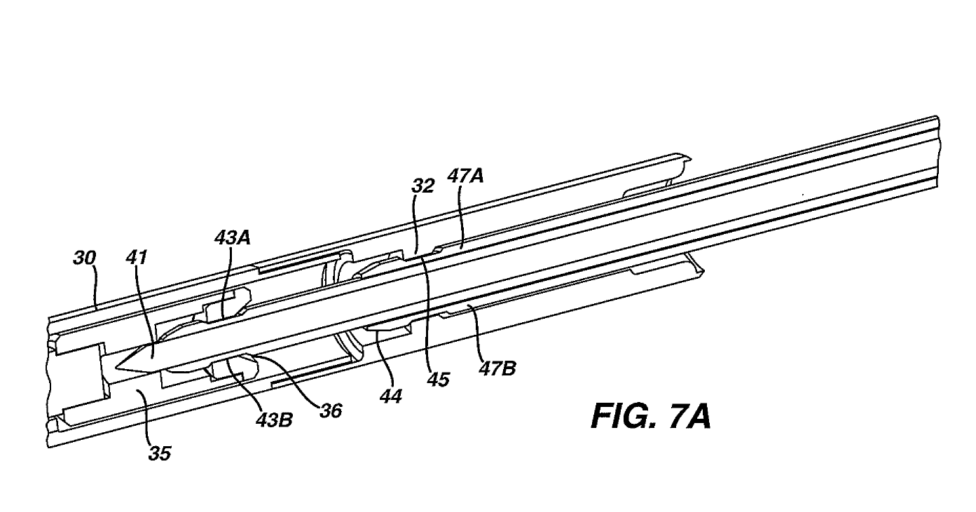

FIG. 7 shows the attachment mechanism (40) attached to the end effector (30). The groove (45) of the shaft arms (47) mates the rib (32) of the end effector (30) preventing relative axial motion. The lateral grooves (43A, 43B) of the second arms (42) mate the ring (33) of the end effector (30) preventing relative axial motion. The rib (32) is rigidly connected to the outer housing (37) of the end effector (30), and the ring (33) is rigidly connected to the jaw actuator (34) via the coupling (35). When the elongate pin (41) is fully advanced, medial deflection of the second arms (42) and the shaft arms (47) is inhibited (see FIG. 7A). Accordingly, axial movement of the arms (42) relative the shaft (22) will cause axial movement of the jaw actuator (34) relative the housing (37), thereby causing the jaws to open and close.

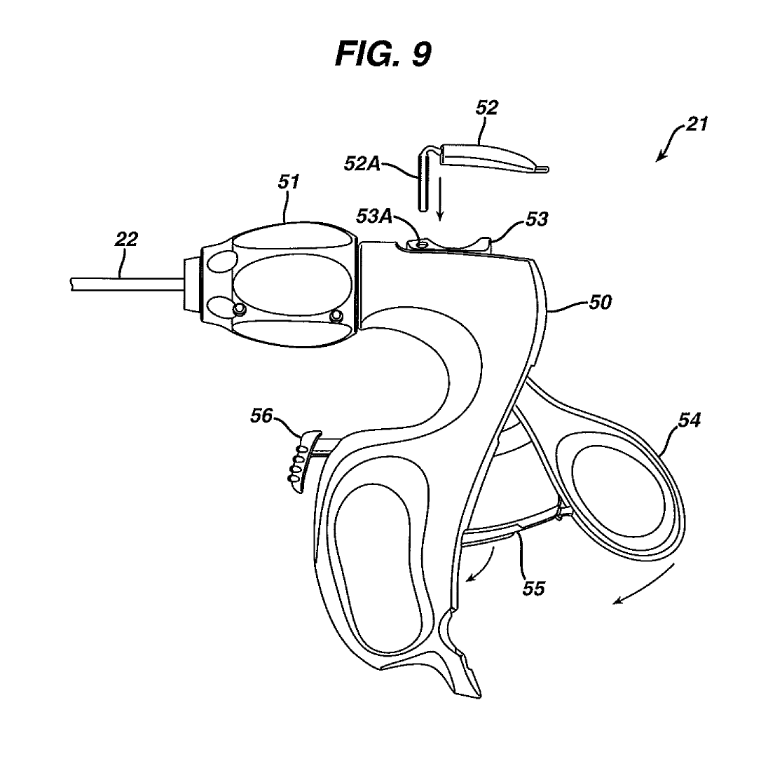

FIG. 9 shows and example of the handle (21) for the instrument (20). The handle (21) includes a base (50). A knob (51) rotates the attachment mechanism (40) about the axis of the shaft (22), which will also rotate an attached end effector (30). The trigger (54) pivots relative the base (50) causing axial movement of the second arms (42) and the pin (41) relative the shaft (22). Operation of the trigger (54) will operate the jaws on an attached end effector (30). The latch (55) pivots relative the base (50) between a locked position (as shown in figure) to prevent operation of the trigger (54) and an unlocked position recessed in the base (50). During seating with the end effector (30), the latch (55) may be locked to maintain the same relative axial spacing of the corresponding the mating features (43, 45) as the mating features (33, 32), resulting in resulting in a single “snap” feedback. The trigger lock (56) can lock/unlock the trigger in/from its depressed position. An actuator (53), which in this embodiment is a slider, controls axial movement of the pin (41) relative the second arms (42). The distal most position of the actuator (53) relative the base (as shown in the figure) places the pin (41) in its locked position, and the proximal most position places the pin (41) in its unlocked position. The pin lock (52) includes a pin (52A) which went inserted into the hole (53A) maintains the pin (41) and second arms (42) in the extended and locked positions as shown in FIG. 5.

The following describes one method for attaching the end effector (30) to the shaft (22). The distal end (23) is introduced in into the proximal end (31) of the end effector (30) with the pin (41) in the unlocked position. The shaft (22) deflects the torque arms (61) laterally into recesses (62) when the torque arms are not aligned with the opening (48). In another expression, torque arm (63) deflects laterally upon shaft (22) insertion into the end effector (30). When the torque arm (61, 63) are aligned with the opening (48), they do not deflect and rest adjacent to opening (48) on the lateral surfaces of shaft arms (47) permitting rotation of the end effector. As the arms (42) are advanced axially into the end effector (30), the chamfered lead (36) of the ring (33) medially deflects the arms (42) until the ring (33) is seated into the lateral notches (43). Simultaneously the shaft arms (47) advance axially into the end effector (30), and the tapered end (44) aligns the rib (32) to seat into the groove (45). In both cases, the surgeon may feel a tactile “click” indicating proper engagement. Once fully seated in the end effector (30), the pin (41) may be slid to the locked position thereby attaching the end effector (30) to the instrument (20). Once attached, the surgeon may pull the end effector from the loader (10), and the loader (10) may then be removed from the surgical field. When the end effector (30) is attached to the shaft (22) and the torque arm (61, 63) are not aligned with the opening (48), the surgeon may grip tissue or another instrument and rotate the knob (51) until the torque arms (61) seat in the opening (48). The surgeon may then manipulate tissue with the end effector (30) as needed for the surgical procedure.

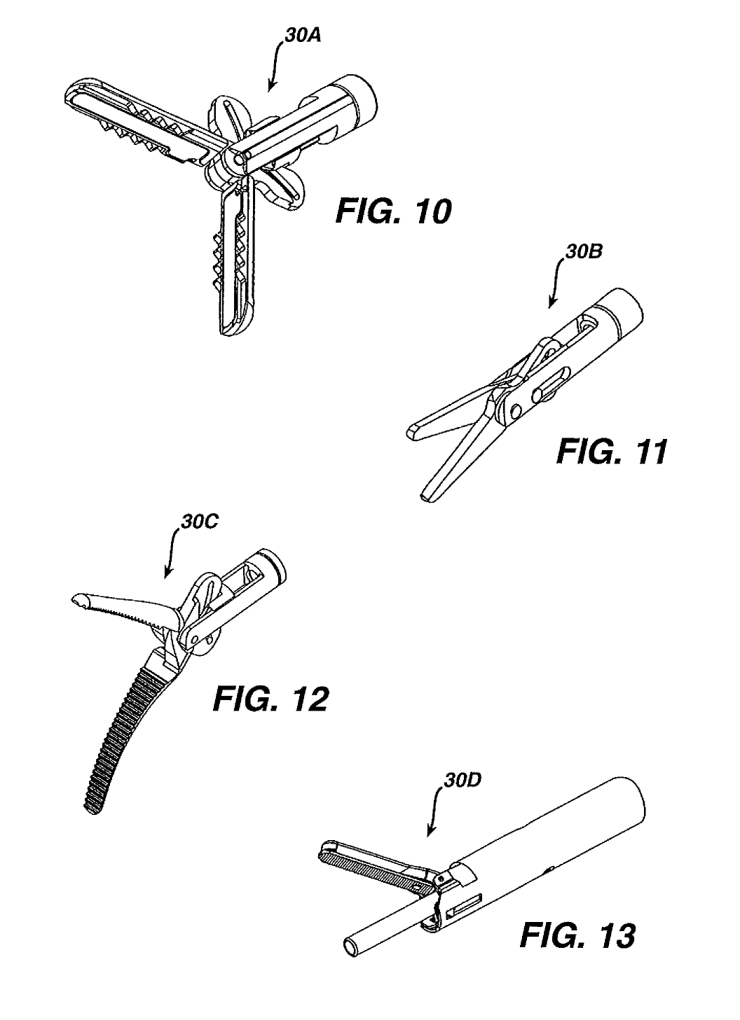

FIGS. 10-13 illustrate some non-limiting examples of alternative end effectors (30A-D) that may attached to the distal end (23) of the instrument (20). In addition to the loader (10) and instrument (20), all or a portion of the end effectors (30, 30A, 30B, 30C, 30D) may be bundled as part of a kit so the surgeon may interchange the attached end effector as needed for a surgical procedure. All the end effectors examples shown here have cooperating jaws; however, non-jawed end effectors could also be employed such as hook knives, snares, and the like. In the case of end effectors that require energy, appropriate energy transmission mechanisms known in the art should be added to the handle (21) and shaft (22). For instance, appropriate electrical connections can be added for the bi-polar forceps end effector (30A). Similarly, an ultrasonic transducer and waveguide can be added for the ultrasonic shears end effector (30D).

The following describes one method for using the devices during a laparoscopic surgical procedure. An instrument (20) is obtained and passed through incision (8). The incision (8) may be a precutaneous incision formed at least partially by a puncture formed with the obtruator on the pin (41) in the configuration shown in FIG. 5. The pin lock (52) and latch (55) may be secured to the slider (53) and trigger (54), respectively. After the puncture, the pin lock (52) may be removed.

A loader (10) and end effector (30) are obtained. The end effector (30) may be selected from a plurality of end effectors provided in a kit. The end effector (30) is loading ex vivo into the distal end (13) of the loader (10). The distal end (13) of the loader (10) with the loaded end effector (30) is passed through incision (4). The second incision (4) may also be percutaneous incision spaced from the first incision (8), and may include passing the distal end (13) with the loaded end effector (30) through a trocar. The distal end (13) may be articulated to facilitate orientation between the proximal end (31) of the end effector (30) and the attachment mechanism (40). The actuator (53) is slid proximally to move the pin (41) to its unlocked position. The distal end (23) of the instrument (20) is advanced into the proximal end (31) of the end effector (30) until the respective mating features of the instrument (20) and end effector (30) are engaged. The actuator (53) may then be slid distally thus advancing the pin (41) to its locked position. The end effector (30) has now been attached in vivo to the instrument (20). The end effector (30) may then be pulled from the loader (10) and the latch (55) disengaged from the trigger (54). Tissue is then manipulating by actuating the trigger (54) of the handle (21) to operate the jaws of the end effector (30).

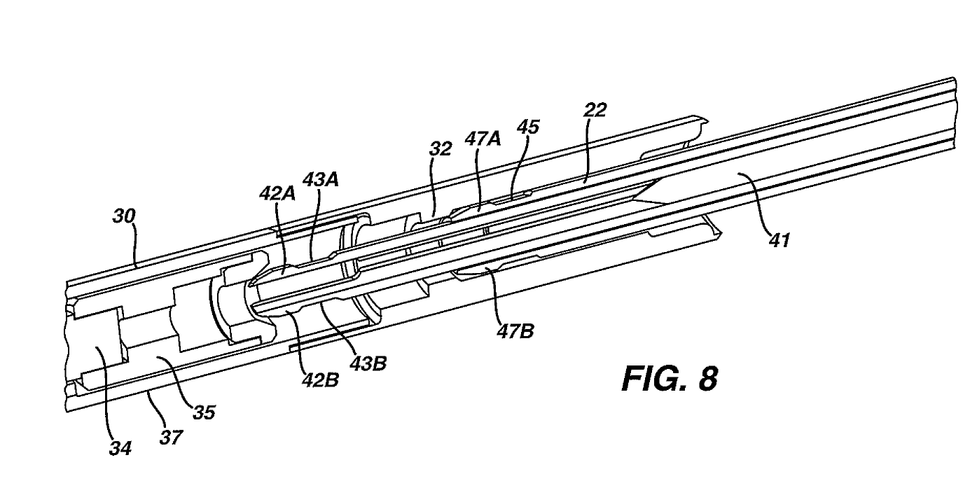

After completing the surgical procedure, the end effector (30) may be detached from the shaft (22). If previously removed, the loader (10) may be reintroduced through the incision (4) into the surgical field. The distal end (32) of the end effector (30) is seated into the distal end (13) of the loader (10), and the pin (41) moved to its unlocked position. The second arms (42) are then proximally withdrawn from the ring (33), deflecting medially as the chamfered portions of the second arms (42) slide over the ring (33) medial surfaces. Accordingly, the device will be in the configuration depicted in FIG. 8. Distally advancing the arms (42) will cause the shaft arms (47) to deflect medially into the opening (48) as the chamfered portions of shaft arms (47) slide over the rib's (32) medial surfaces which simultaneously cause the second arms (42) to deflect medially into the gap (46) facilitating easier separation of the end effector (30) from the shaft (22). The distal advancement of the shaft (22) continues until the rib (32) unseats from the groove (45). This unseating may be facilitated by the jaws of the end effector (30) being held in a closed position by the tube in the loader distal end (13). The distal end (23) may then be withdrawn from the end effector (30) thus detaching the end effector (30) from the instrument (20). The end effector will be held in the loader (10) by virtue of the engagement feature (16). Removal of the loader (10) from the surgical field will remove the end effector (30). A different end effector may then be attached to the instrument (20), or the instrument (20) may be withdrawn from the surgical field.

Without limitation, the following describe some of the benefits and advantages of the foregoing devices and methods over the prior art. The end effector (30) may have a much larger diameter than the shaft (22); accordingly, the incision (8) can be smaller compared to more traditional laparoscopic instruments resulting in less pain and scarring, and quicker recovery. This also facilitates a small diameter shaft (22) (even less than 3 mm), thus potentially eliminating a trocar in the incision (8). The attachment mechanism (40) provides quick end effector (30) exchanges with the instrument (20), thus decreasing surgical time. The loader (10) also facilitates quick end effector (30) exchanges. A kit of multiple end effectors may reduce instrument costs by consolidating a single shaft (22) and handle (21) for all instruments. Many other benefits will be apparent to those skilled in the art.

Having shown and described various embodiments and examples of the present invention, further adaptations of the methods and devices described herein can be accomplished by appropriate modifications by one of ordinary skill in the art without departing from the scope of the present invention. Several of such potential modifications have been mentioned, and others will be apparent to those skilled in the art. For instance, the specific materials, dimensions, and the scale of drawings will be understood to be non-limiting examples. Accordingly, the scope of the present invention should be considered in terms of the following claims and is understood not to be limited to the details of structure, materials, or acts shown and described in the specification and drawings.

1. A surgical device, comprising:
a) an elongate shaft defining a longitudinal axis, the shaft comprising a distal end and a proximal end;
b) an first arm on the elongate shaft comprising a mating feature, the first arm being medially deflectable;
c) a second arm longitudinally spaced from the first arm comprising a mating feature, the arm being axially slideable relative the elongate shaft and being medially deflectable;
d) an elongate pin positioned medially relative the second arm, the elongate pin being axially slideable relative the first arm and second arm between a locked position preventing medial deflection of the first arm and second arm and an unlocked position allowing medial deflection of the first arm and second arm; and
e) a surgical end effector selectively attachable and detachable to the mating feature of the second arm.

2. The surgical device of claim 1, further comprising a lateral notch on the distal end of the first arm and a mating feature on the surgical end effector.

3. The surgical device of claim 2, wherein the mating feature comprises a ring dimensioned to mate with the first arm lateral notch.

4. The surgical device of claim 1, comprising two or more second arms circumscribing the elongate pin.

5. The surgical device of claim 1, comprising two or more first arms.

6. The surgical device of claim 5, wherein the opening is defined by the two or more first arms.

7. The surgical device of claim 1, wherein the distal end of the elongate pin comprises an obtruator tip.

8. The surgical device of claim 1, further comprising a handle operatively connected to the proximal end of the elongate shaft, the handle comprising a trigger controlling the axial movement of the second arm and an actuator controlling the axial movement of the elongate pin.

9. The surgical device of claim 8, wherein the actuator is lockable.

10. The surgical device of claim 8, wherein the end effector has opposable members that move between open and closed positions in response to axial motion of the second arm.

11. A laparoscopic surgical device, comprising:
a) an elongate shaft defining a longitudinal axis, the shaft comprising a distal end and a proximal end;
b) a plurality of first arms on the distal end of the shaft, the first arms each comprising a lateral notch, the arms being medially deflectable;
c) a plurality of second arms positioned medial to the first arms, the second arms projecting distally from the distal end of the elongate shaft and being longitudinally spaced from the first arms, the second arms each comprising a lateral notch, the second arms being axially slideable relative the elongate shaft and being medially deflectable;
d) an elongate pin positioned medially relative the second arms, the elongate pin being axially slideable relative the arms between a locked position preventing medial deflection of the arms and an unlocked position allowing medial deflection of the arms; and
e) a surgical end effector selectively attachable in vivo and detachable in vivo to the mating features of the first arms and the second arms.

12. The laparoscopic surgical device of claim 11, further comprising an obtruator tip on the distal end of the elongate pin.

13. The laparoscopic surgical device of claim 11, wherein the plurality of first arms defines an opening in the elongate shaft.

14. The laparoscopic surgical device of claim 13, wherein the opening extends from the shaft lateral surface through the shaft medial surface.

15. The surgical device of claim 11, further comprising a handle operatively connected to the proximal end of the elongate shaft, the handle comprising a trigger controlling the axial movement of the second arm and an actuator controlling the axial movement of the elongate pin.

16. A laparoscopic surgical device, comprising:
a) an elongate shaft defining a longitudinal axis, the shaft comprising a distal end and a proximal end;
b) a plurality of first arms on the distal end of the shaft, the first arms each comprising a lateral notch, the arms being medially deflectable, the arms defining an opening in the shaft;
d) a plurality of second arms positioned medial to the first arms, the second arms projecting distally from the distal end of the elongate shaft and being longitudinally spaced from the first arms, the second arms each comprising a lateral notch, the second arms being axially slideable relative the elongate shaft and being medially deflectable;
d) an elongate pin positioned medially relative the second arms, the elongate pin being axially slideable relative the arms between a locked position preventing medial deflection of the arms and an unlocked position allowing medial deflection of the arms, the pin having an obturator tip;
e) a surgical end effector selectively attachable in vivo and detachable in vivo to the mating features of the first arms and the second arms, the surgical end effector comprising a means to manipulate tissue, the tissue manipulation means actuated in response to the axial movement of the second arms when attached to the surgical end effector; and
f) a handle operatively connected to the proximal end of the elongate shaft, the handle comprising a trigger controlling the axial movement of the second arm and an actuator controlling the axial movement of the elongate pin.
